(19) United States
(12) Patent Application Publication
(10) Pub. No.: US 2024/0003882 A1
(43) Pub. Date: Jan. 4, 2024

(54) DEVELOPMENT OF A NOVEL ELISA-BASED POINT-OF-CARE ANTIGEN TEST FOR SARS-COV-2

(71) Applicant: The Regents of the University of California, Oakland, CA (US)

(72) Inventors: Yusaku Michael Shino, Los Angeles, CA (US); Airi Harui, Culver City, CA (US)

(73) Assignee: The Regents of the University of California, Oakland, CA (US)

(21) Appl. No.: 18/252,605

(22) PCT Filed: Nov. 18, 2021
(86) PCT No.: PCT/US21/59934

§ 371 Date: May 11, 2023

Related U.S. Application Data
(60) Provisional application No. 63/236,006, filed on Aug. 23, 2021.
(60) Provisional application No. 63/115,164, filed on Nov. 18, 2020.

(51) Int. Cl. G01N 33/569; C07K 16/10

(52) U.S. Cl. CPC G01N 33/56983; C07K 16/1003; G01N 2333/165

(57) ABSTRACT

Aspects of the present disclosure relate to methods and systems designed to utilize antibodies and viral inactivating/lysis agents to detect SARS-CoV-2 on a microfluidic channel-based platform, for example in samples obtained from nasal swabs taken from infected individuals. The rapid and accurate point of care COVID-19 diagnostic tests that are disclosed herein can transform medical professional's testing capabilities, and in this way improve the ability to control the COVID-19 pandemic.

### CROSS-REFERENCE TO RELATED APPLICATIONS

This application claims the benefit under 35 U.S.C. Section 119(e) of co-pending U.S. Provisional Patent Application Ser. No. 63/115,164 filed on Nov. 18, 2020, and co-pending U.S. Provisional Patent Application Ser. No. 63/236,006, filed on Aug. 23, 2021, which applications are incorporated in their entirety by reference herein.

### TECHNICAL FIELD

Embodiments of the disclosure concern at least the fields of immunology, virology and medicine.

### BACKGROUND OF THE INVENTION

Almost two years into the COVID-19 pandemic, our rapid SARS-CoV-2 testing capabilities remain limited. Laboratory based reverse transcription polymerase chain reaction (RT-PCR) tests that amplify viral RNA are the gold standard for SARS-CoV-2 detection. However, these tests are labor intensive, require specialized equipment, trained personnel and transportation to a centralized laboratory. Currently, there are two types of rapid SARS-CoV-2 tests with emergency use authorizations (EUA) from the FDA: 1) rapid antigen tests and 2) rapid molecular tests. Rapid antigen tests that measure viral proteins using lateral flow assay technology are limited by poor sensitivity, especially in patients with lower viral concentrations (1-4). Rapid molecular tests that amplify and measure viral RNA have better sensitivity but are limited by low throughput of only a few samples per hour.

There is a need in the art for the development of rapid and accurate point-of-care COVID-19 diagnostic tests with high-throughput as this will transform the current testing capabilities and improve the ability to control the pandemic.

### SUMMARY OF THE INVENTION

As discussed below, we developed, optimized and validated a novel ELISA-based assay for the detection of SARS-CoV-2 antigen, one that exhibits superior performance to existing antigen tests. Typically in the assays disclosed herein, selected antibodies are used in combination with an optimized media that includes one or more viral inactivating agents. The assays can be further optimized using a microfluidic assay platform having microfluid channels for viral detection. Such assay platforms offers several advantages including: improved sensitivity compared with existing rapid antigen tests, improved throughput compared with rapid molecular tests, and an automated point-of-care platform which can be deployable in remote locations without the need for a centralized laboratory. Furthermore, results of the assays disclosed herein can be reported both qualitatively and quantitatively as viral RNA copies per milliliter.

In illustrative embodiments of the invention, the assay is performed on an antigen assay platform that comprises microfluidic channels disposed in a cartridge, and the samples and reagents are directed by pneumatic pistons and valves through these channels where three sandwich ELISAs are detected within each microfluidic channel. This microfluidic design and triplicate detection improves both the sensitivity and precision of SARS-CoV-2 measurement at low antigen concentrations. Importantly, SARS-CoV-2 detection can be reported both qualitatively (positive/negative/indeterminate) and quantitatively (viral RNA copies per milliliters and/or N protein levels) making this assay the first SARS-CoV-2 test capable of reporting viral concentrations. Notably, the viral quantification in this assay is volume insensitive (i.e. the sample volume used in the assay does not affect concentration measurements).

Embodiments of the invention are optimized for targeting SARS-CoV-2 virus (including multiple COVID variants) and include, for example, combinations of selected antibodies targeting SARS-CoV-2 proteins (e.g., the SARS-CoV-2 S, M, N and/or E proteins). Embodiments of the invention further include, for example, what we have discovered to be optimized antibody combinations, concentrations, optimized sample collection and viral inactivating reagents, as well as optimized sample processing and method steps. Embodiments of the invention include SARS-CoV-2 antigen tests having superior speed and throughput as compared with conventional SARS-CoV-2 antigen tests. For example, certain embodiments of the invention are designed to detect at least 72 samples per 80 minutes.

Embodiments of the invention also include, for example, methods of detecting the presence of SARS-CoV-2 in a fluid sample (e.g., nasopharyngeal swab samples and the like). These methods typically comprise combining the fluid sample with an immobilized capture antibody that binds SARS-CoV-2 peptide within the microfluidic channel. A detection antibody is then combined with and will bind to the SARS-CoV-2 peptide “captured” within the channel. The amount of detection antibody bound to the SARS-CoV-2 peptide within the microfluidic channel is then quantified by methods well known in the art. For example, embodiments of the invention can include a biotinylated detection antibody to detect SARS-CoV-2 peptide. In such embodiments, streptavidin coupled to a label such as a fluorochrome will then bind the biotin and the amount of fluorochrome bound within the microfluidic channel can be quantified by a detector. Other embodiments of the invention can include other labels bound directly or indirectly to the detection antibody, such as enzymes, nucleic acids or other analytes, which can be quantified using art accepted methods. In these methods, the capture antibody and the detection antibody are typically selected from the antibodies that bind to SARS-CoV-2 peptides within the microfluidic channel with varying sensitivities and specificities as described herein (e.g., in the Tables below). In certain embodiments of these methods, the capture antibody and the detection antibody include antibodies designated as follows: N6 antibody, N7 antibody, N5 antibody and/or N11. For example, in certain embodiments of the invention, N7 is the capture antibody and N11 is the detection antibody; and/or N6 is the capture antibody and N11 is the detection antibody; and/or N6 is the capture antibody and N5 is the detection antibody. In certain embodiments of the invention, these capture and detection antibodies are transposed, for example so that N11 is the capture antibody and N7 is the detection antibody.

In typical embodiments of the invention, the fluid test sample is combined with an agent or agents selected to inactivate the SARS-CoV-2 prior to combining the virus with the antibodies. Optionally, for example, the fluid sample is combined with a nonionic surfactant (e.g., one that has a hydrophilic polyethylene oxide chain and an aromatic hydrocarbon lipophilic or hydrophobic group such as triton-X nonionic surfactant) so as to inactivate SARS-CoV-2 prior to detecting the presence of SARS-CoV-2 in the fluid sample. Other embodiments of the invention can inactivate the SARS-CoV-2 virus with detergents such as Tween 20.

In some embodiments of the invention, the methods are designed to detect less than 25,000 SARS-CoV-2 RNA copies per milliliter in the fluid sample. In typical embodiments of the invention, the methods are performed in an assay device comprising microfluidic channels. In such embodiments, the fluid samples and reagents are directed through the microfluidic channels by pneumatic pistons and valves and the sandwich ELISA reactions are quantified within the channel. Some embodiments of the invention can detect three sandwich ELISAs where N7 is the capture antibody and N11 is the detection antibody; and/or N6 is the capture antibody and N11 is the detection antibody; and/or N6 is the capture antibody and N5 is the detection antibody. In certain embodiments of the invention, the detection antibody is present in concentrations between 0.5 μg/mL and 200 μg/mL and the capture antibody is present in concentrations between 0.5 μg/mL and 200 μg/mL.

Embodiments of the invention also include systems and kits for detecting the presence of SARS-CoV-2 in a fluid sample. In illustrative embodiments, the system or kit comprises one or more capture antibodies which binds to a SARS-CoV-2 peptide; and further includes one or more detection antibodies that is coupled or couplable to a detectable label. In such embodiments of the invention, the capture antibody and the detection antibody are selected from the antibodies that bind to epitopes on the SARS-CoV-2 bound by antibodies identified herein (e.g., in the Tables below). In illustrative working embodiments of the invention, the capture antibody and the detection antibodies N6 antibody, N7 antibody, N5 antibody and/or N11 antibody. For example, in certain embodiments of the invention, N7 is the capture antibody and N11 is the detection antibody; and/or N6 is the capture antibody and N11 is the detection antibody; and/or N6 is the capture antibody and N5 is the detection antibody. In some embodiments of the invention, the system or kit comprises for example an assay device (e.g., a cassette or a cartridge) that includes microfluidic channels and the capture and detection antibodies are coupled to one or more regions in the microfluidic channels. Typically, the fluid samples and reagents are directed in the assay device by pneumatic pistons and valves through the microfluidic channels wherein the microfluidic channels comprise ELISAs.

Embodiments of the assays disclosed herein can improve upon the currently available antigen tests in at least three significant ways. First leveraging the superior performance characteristics of microfluidic assays (e.g., the Ella Protein Simple platform), will improve both sensitivity and precision. Second, leveraging an automated platform that can run multiple samples concurrently without any overlapping channels, will improve test throughput without any cross-contamination of samples. Third, by running a standard curve of SARS-CoV-2 quantified by droplet digital PCR, the test will report results in SARS-CoV-2 viral RNA copies/mL as well as N protein levels. Embodiments of the invention provide the first quantitative diagnostic test capable of resulting viral concentrations.

As noted above, we have developed a novel rapid point-of-care SARS-CoV-2 antigen test. The test can provide accurate and high-throughput testing in many remote locations without access to laboratory-based RT-PCR (remote clinics, developing countries, cruise/military ships). Furthermore, the ability to determine viral concentrations will catalyze novel studies that may deepen our understanding of viral transmission, disease progression and prognosis. Furthermore, the disclosure provided herein regarding the optimal antibodies and antibody concentrations used for SARS-CoV-2 detection is transferable to both lateral flow assays as well as a wide variety of ELISA assays.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Schematic showing an illustrative antigen assay platforms. FIG. 1 shows an Ella Protein Simple assay device with a 72-sample cartridge. The left panel provides a photograph of the microfluidic assay platform, the middle panel provides an enlarged drawing of the microfluidic assay cartridge, the right panel provides views of the microfluidic channels where “glass nanoreactors” measure three sandwich ELISAs per channel detect SARS-CoV-2 peptides.

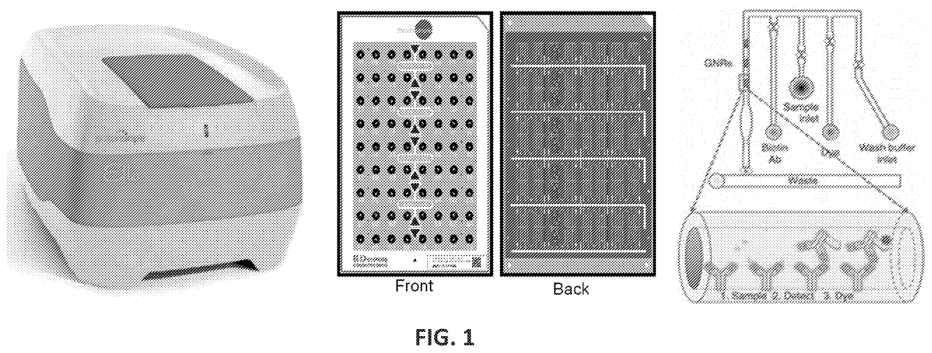

FIG. 2. Schematic showing illustrative samples and reagents directed through “microfluidic channels” by pneumatic pistons and valves. FIG. 2 provides views of three “glass nanoreactors” designed to capture three sandwich ELISAs per channel (this schematic is for a 16-sample 4-analyte cartridge). The left panel provides a photograph of the microfluidic assay platform, the middle panel provides an enlarged drawing of the microfluidic assay platform, and the right panel provides a further enlarged drawing of the microfluidic assay platform.

FIG. 3. Schematic showing a 48-sample “Open cartridge” embodiment which allows for the addition of any combination of sample, capture and detection antibodies to each well.

FIG. 4. Graphed data from studies using N7 as the capture antibody and N11 as the detection antibody. With this embodiment of the invention, the limit of detection is 25,000 RNA copies/mL with an RFU signal cutoff of 0.28. In comparison, conventional lateral flow assays have been shown to have limits of detection in the hundreds of thousand RNA copies/mL (1-4). As depicted in gamma-irradiated SARS-CoV-2 Standard curves, there is a linear relationship between viral concentration and RFU for viral concentrations less than 500,000 RNA copies/mL; Viral concentration=114572*RFU−7027.5. At concentrations above 500,000 the standard curve is upward sloping: Viral concentration=124.18*(RFU)\hat{ }+151677*RFU−460547.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

The disclosure provided herein includes immunoassays that provide elegant, unique and very powerful designs which significantly improve the performance of ELISA assays directed to COVID-19 antigens. With such assay designs, we provide a rapid, sensitive and precise ELISA antigen test with several advantages over existing rapid tests. For example, lateral flow ELISA assays are estimated to have limits of detection for SARS-CoV-2 virus in the hundreds of thousands of viral RNA copies per milliliter. In contrast, gold standard PCR tests have limits of detection of around 6000 RNA copies per milliliter based on the FDA reference panel. As discussed below, embodiments of the invention disclosed herein unexpectedly allow for the detection of SARS-CoV-2 at concentrations less than 25,000 SARS-CoV-2 viral RNA particles per milliliter, 10,000 SARS-CoV-2 viral RNA particles per milliliter or even 5,000 SARS-CoV-2 viral RNA particles per milliliter. Thus, such embodiments have sensitivities competitive with RT-PCR, but as a full automated point-of-care test, with minimal sample processing and labor required.

Embodiments of the invention disclosed herein fill a significant void in the current SARS-CoV-2 testing scheme and provide three distinct advantages: 1) Improved sensitivity and precision compared with existing rapid tests, 2) Increased throughput compared with existing point-of-care RT-PCR tests, and 3) Quantitative resulting: in the disclosed assays being the first quantitative SARS-CoV-2 diagnostic test able to report as viral concentration and or N protein levels. The ability to determine a patient's viral concentration will improve our understanding of viral transmission and disease progression. This test will also be useful in remote locations without access to RT-PCR labs such as developing countries, rural communities, cruise/military ships. In addition, the disclosed assay designs are transferable to other ELISA-based systems (e.g., automated ELISA and lateral flow assays) and can therefore improve the sensitivity of detecting any virus of interest. Consequently, we can apply the principles described above to develop antigen tests for other viruses including influenza, parainfluenza and RSV—all of which continue to cause significant morbidity and mortality every year.

In our studies of SARS-CoV-2 antigen detection, we tested over 2000 combinations of different Ab pairs, sample concentrations and assay conditions. As part of this, we screened 40 commercially available antibodies from 6 companies in numerous combinations (as single and “mixed antibodies”), concentrations and conditions. The manufacturer of these antibodies does not provide affinity data, so we evaluated the antibodies on the Ella Simple microfluidic platform using recombinant N-protein, S-protein and gamma-irradiated virus. (Table 2)

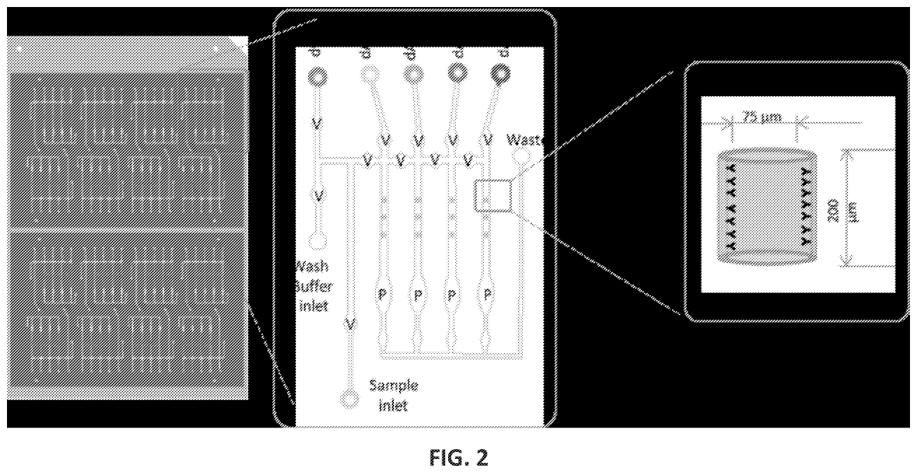

In these studies, we discovered that there is significant variability in the affinity and sensitivity of these antibodies to the recombinant proteins, as well as inactivated virus. The different antibody concentrations and assay conditions also make a large difference in the results. After significant trial and error, we have optimized the assay in terms of sample (e.g., nasopharyngeal swab samples or the like) processing, antibody pairs/concentrations, antibody labeling and assay reagents including inactivating agents. Furthermore, we have discovered that S-protein antibodies had poor binding to inactivated virus. We hypothesize that this is due to lysing of the virus during our processing which could make the S-protein denatured and/not available for antibody binding. We evaluated numerous inactivation methods (UV, heat inactivation and combination of inactivating agents (PFA+Triton-X) and detergents) as well as sample collection media for optimal SARS-CoV-2 antigen detection. In illustrative embodiments of the invention, the assay is performed in an antigen assay platform that comprises microfluidic channels disposed in a cartridge, and the samples and reagents are directed by pneumatic pistons and valves through these channels where three sandwich ELISAs per channel (see, e.g., FIG. 2) detect the amount of SARS-CoV-2 peptide present. Thus, each peptide is detected in triplicate, thereby improving both precision and sensitivity of the measurement at low antigen concentrations.

As noted above, we tested over 2000 combinations of different Ab pairs, sample concentrations and assay conditions. From our studies we have discovered that the antibody combinations of N6 antibody, N7 antibody, N11 antibody and/or N5 antibody provide surprisingly low backgrounds with increased SARS-CoV-2 signals. Unexpectedly, in embodiments of the invention where N7 is the capture antibody (“N7C”) and N11 is the detection antibody (“N11D”); and/or embodiments of the invention where N6 is the capture antibody (“N6C”) and N11 is the detection antibody (“N11 D”); and/or embodiments of the invention where N6 is the capture antibody (“N6C”) and N5 is the detection antibody. (“N5D”), the methods using these combination of antibodies can precisely and reproducibly detect SARS-CoV-2 present in the sample at concentrations of 25,000 RNA copies per milliliter.

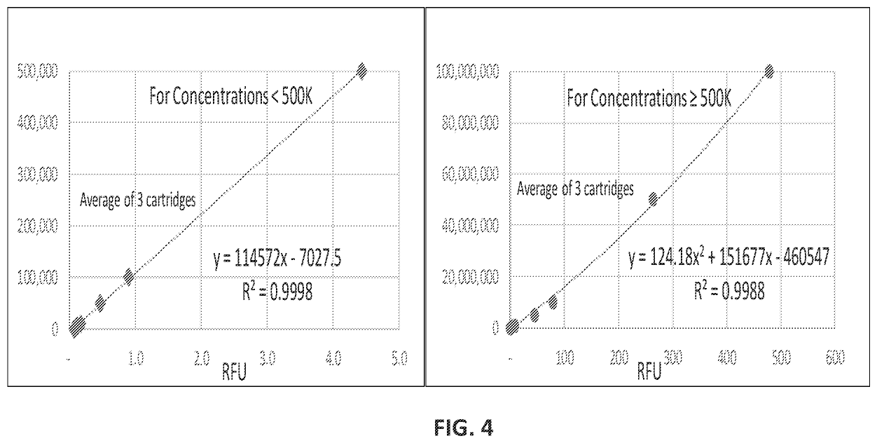

The antibodies used in the assays disclosed herein are well known in the art. N5 is the N-protein antibody 40143-R040 (monoclonal rabbit IgG Clone #040) from Sino Biologicals. N6 is the N-protein antibody MAB10474 (monoclonal Mouse IgG2B Clone #1035111) from R&D. N7 is the N-protein antibody MAB104741 (monoclonal mouse IgG1 Clone #1035145) from R&D. N11 is the N protein antibody. MAB104742 (R&D Systems Clone 10351430. One optimal antibody pairing comprises using N7 as the capture antibody and N11 as the detection antibody, for example at concentrations of 3.5 ug/mL and 0.4 ug/mL, respectively. This antibody pair has consistently provided good signals with low background. With this embodiment of the invention, the limit of detection can be 25,000 RNA copies/mL with an RFU signal cutoff of 0.28. (FIG. 4). In comparison, conventional lateral flow assays have been shown to have limits of detection in the hundreds of thousand RNA copies/mL (1-4). As depicted in gamma-irradiated SARS-CoV-2 Standard curve (FIG. 4), there is a linear relationship between viral concentration and RFU for viral concentrations less than 500,000 RNA copies/mL; Viral concentration=114572*RFU−7027.5. At concentrations above 500,000 the standard curve is upward sloping: Viral concentration=124.18*(RFU)\hat{ }2+151677*RFU−460547.

Embodiments of the invention are adapted for SARS-CoV-2 sample collection from nasal swabs, oral swabs, saliva and the like. In an exemplary nasal swabbing protocol, one can: (1) insert nasal swab 1.5 inches into nostril and turn 5 times; (2) repeat in other nostril: 1.5 inches into nostril and turn 5 times; and then (3) place swab in collection tube with optimized media—plunge swab up and down while twisting back and forth for 60 seconds to release virus into media. A variety of medias can be used in such assays. For example, an exemplary media formulation comprises 0.1% of Triton X and 0.05% Tween 20 added into 1× (R&D) reagent diluent No. 895182.

We screened 40 antibodies from 6 companies at various combinations and concentrations. We discovered significant variability of the antibody affinity to the SARS-CoV-2 peptides and background noise between antibodies. We evaluated over 2000 combinations of antibodies under different assay conditions and optimized the assay to detect SARS-CoV-2 antigen with the best signal to noise ratio, precision and reproducibility. We optimized the assays to maximize the linearity of the standard curve at lower viral concentrations with minimal variability. Patients with high SARS-CoV-2 viral loads at the time of their nasal swabs will have high RFUs and be straightforward diagnostically. Patients with lower viral loads (<100,000 RNA copies/mL) are more challenging diagnostically, since the RFUs will be lower with higher coefficients of variation. Thus, the assay was optimized with a focus on accuracy and precision at the lower viral concentrations. For example, we discovered that increasing the capture or detection antibody concentration increased the detection signal, but also increases the background noise, thus often leading to decreased signal to noise ratios Decreasing the capture or detection antibody concentrations decreased the background signal, but also decreased the detection signal. Thus, the antibody concentrations were optimized to maximize the signal to noise ratio at lower viral concentrations.

We evaluated various methods of viral inactivation including the use of paraformaldehyde (PFA) at various concentrations (1, 2, 4%), formalin (5%, 10⁰/), triton-X 0.1%. We find that triton-X at about 0.1% gives the fastest, most reliable method of inactivation while keeping the background low. Paraformaldehyde at 1% provided good signal to noise ratio—but the higher paraformaldehyde and formalin solutions provided poor signals. In this context, embodiments of the invention include assay media comprising triton X and Tween 20. For example, embodiments of the invention include an assay media comprising about 0.1% Triton X and about 0.05% Tween 20. Optionally, the assay media comprises the base media of R&D System Part #895182.

We have also evaluated various concentrations for the labeling of digoxigenin to the capture antibodies and biotin to the detection antibodies. We find that 5:1 molar excess of digoxigenin to antibody and 10:1 molar excess of biotin to antibody (as recommended by Ella Protein Simple as a starting point) provided the best signal to noise ratio in the assay. We also evaluated several diluents which was used to dilute the samples and antibodies (in combination). We evaluated the following sample diluents from Ella Protein Simple: SD06 #896096, SD10 #896097, SD13 #896098).

Methods, materials and systems that can be adapted for performing aspects of the claims invention are disclosed in U.S. Patent Application Publication Nos.: 20200291480, 20200277643, 20200200707, 20200155966, 20200062768, 20190369068, 20190369048, 20190285634, 20190257827, 20190248885, 20190218531, 20180321189, 20170275700, 20170151564, 20170065978, 20170050186, 20170001197, 20160370319, 20160230203, 20150241389, 20140106372, 20130167937, and 20120213667; as well as Oliveira et al., Rev Inst Med Trop Sao Paulo 2020 Jun. 29; 62:e44. doi: 10.1590/S1678-9946202062044; Kohmer et al., J Clin Virol. 2020 August; 129:104480. doi: 10.1016/j.jcv.2020.104480. Epub 2020; and Wang et al., Emerg Microbes Infect. 2020 December; 9(1):2200-2211. doi: 10.1080/22221751.2020.1826362; the contents of all of which are incorporated herein by reference.

Embodiments of the invention include, for example, methods of detecting the presence of SARS-CoV-2 in a fluid sample (e.g., nasopharyngeal swab samples and the like). These methods typically comprise combining the fluid sample with an immobilized capture antibody that binds SARS-CoV-2 peptide within the microfluidic channel. A detection antibody is then combined and will bind to the SARS-CoV-2 peptide “captured” within the channel. The amount of detection antibody bound to the SARS-CoV-2 peptide within the microfluidic channel is then quantified according to art accepted methods. Embodiments of the invention can include a biotinylated detection antibody. Streptavidin coupled to a label such as a fluorochrome will then bind the biotin and the amount of fluorochrome bound within the microfluidic channel will be quantified by a detector. Other embodiments of the invention can include other labels bound directly or indirectly to the detection antibody, such as enzymes, nucleic acids or other analytes, which can be quantified according to art accepted methods. In these methods, the capture antibody and the detection antibody are typically selected from the antibodies that bind to SARS-CoV-2 peptides within the microfluidic channel and which have the epitope targets, sensitivities and/or specificities as described herein (e.g., in the Tables below). In this context, epitope mapping studies that allow the identification of such epitopes are well known in the art (see, e.g., U.S. Patent Application Publication Nos.: 20170196971; 20170131276; 20110071043; and 20100136030, the contents of which are incorporated herein by reference). In certain embodiments of the invention, the antibodies are selected to have an affinity for such epitopes on SARS-CoV-2 that is at least equal to the affinity exhibited by the antibodies identified herein (e.g., N5 antibody, N6 antibody, N7 antibody or N11 antibody). In certain embodiments of the invention, the antibodies are selected to have a sensitivity for such epitopes on SARS-CoV-2 that is at least equal to the affinity exhibited by the antibodies identified herein (e.g., N5 antibody, N6 antibody, N7 antibody or N11 antibody).

In certain embodiments of these methods, the capture antibody and the detection antibody include antibodies designated as follows: N6 antibody, N7 antibody, N5 antibody and/or N11. For example, in certain embodiments of the invention, N7 is the capture antibody and N11 is the detection antibody; and/or N6 is the capture antibody and N11 is the detection antibody; and/or N6 is the capture antibody and N5 is the detection antibody. In certain embodiments of the invention, these capture and detection antibodies are transposed, for example so that N11 is the capture antibody and N7 is the detection antibody, so that N11 is the capture antibody and N6 is the detection antibody, or so that N5 is the capture antibody and N6 is the detection antibody. In typical embodiments of the invention, the fluid sample is combined with an agent or agents selected to inactivate the SARS-CoV-2 prior to combining the virus with the antibodies. Optionally, for example, the fluid sample is combined with a nonionic surfactant (e.g., one that has a hydrophilic polyethylene oxide chain and an aromatic hydrocarbon lipophilic or hydrophobic group such as triton-X nonionic surfactant) so as to inactivate SARS-CoV-2 prior to detecting the presence of SARS-CoV-2 in the fluid sample. Other embodiments of the invention will inactivate the SARS-CoV-2 virus with detergents such as Tween 20.

In some embodiments of the invention, the methods are designed to detect 25,000 SARS-CoV-2 viral RNA particles per milliliter with a relative fluorescence unit (RFU) intensity of 0.28. In typical embodiments of the invention, the methods are performed in an assay device comprising microfluidic channels. The fluid samples and reagents are directed through the microfluidic channels by pneumatic pistons and valves and the sandwich ELISAs are quantified within the channel. Some embodiments of the invention will detect three sandwich ELISAs where N7 is the capture antibody and N11 is the detection antibody; and/or N6 is the capture antibody and N11 is the detection antibody; and/or N6 is the capture antibody and N5 is the detection antibody. In certain embodiments of the invention, the detection antibody is present in concentrations between 0.5 μg/mL and 200 μg/mL and the capture antibody is present in concentrations between 0.5 μg/mL and 200 μg/mL. In some embodiments of the invention, the detection antibody is present in concentrations between 0.5 μg/mL and 10 μg/mL (e.g., between 1-5 μg/mL, between 2-3.5 μg/mL etc.) and the capture antibody is present in concentrations between 0.5 μg/mL and 10 μg/mL (e.g. between 1-5 μg/mL, between 2-3.5 μg/mL, about 1 μg/mL etc.).

Embodiments of the invention also include systems and kits for detecting the presence of SARS-CoV-2 in a fluid sample. In illustrative embodiments, the system or kit comprises a capture antibody that is coupled to a matrix and binds to a SARS-CoV-2 peptide; and further includes a detection antibody that is coupled or couplable to a detectable label. In such embodiments of the invention, the capture antibody and the detection antibody are selected from the antibodies that bind to epitopes on the SARS-CoV-2 bound by antibodies identified herein (e.g., in the Tables below). In illustrative working embodiments of the invention, the capture antibody and the detection antibodies N6 antibody, N7 antibody, N5 antibody and/or N11 antibody. For example, in certain embodiments of the invention, N7 is the capture antibody and N11 is the detection antibody; and/or N6 is the capture antibody and N11 is the detection antibody; and/or N6 is the capture antibody and N5 is the detection antibody. In some embodiments of the invention, the system or kit comprises for example an assay device (e.g., a cassette or a cartridge) that includes microfluidic channels and the capture antibody is coupled to one or more regions in the microfluidic channels. Typically, the fluid samples and reagents are directed in the assay device by pneumatic pistons and valves through the microfluidic channels wherein the microfluidic channels comprise ELISAs.

As noted above, embodiments of the invention include optimized and validated ELISA-based point-of-care antigen test for SARS-CoV-2 using an existing research use only platform named Ella ProteinSimple (Ella®). Ella is a rapid, fully automated immunoassay platform that can run up to 72 samples loaded on a single cartridge in 80 mins with no additional labor required. The technology uses pneumatic pistons and values to direct the sample through microfluidic channels and three “glass nanoreactors” where three sandwich ELISAs are captured per sample. Thus, each analyte is detected in triplicate, improving both precision and sensitivity, compared with standard ELISA. Importantly, there is no exposed waste generated from the assay. All reagent, sample and diluent waste is contained in a sealed chamber inside the cartridge.

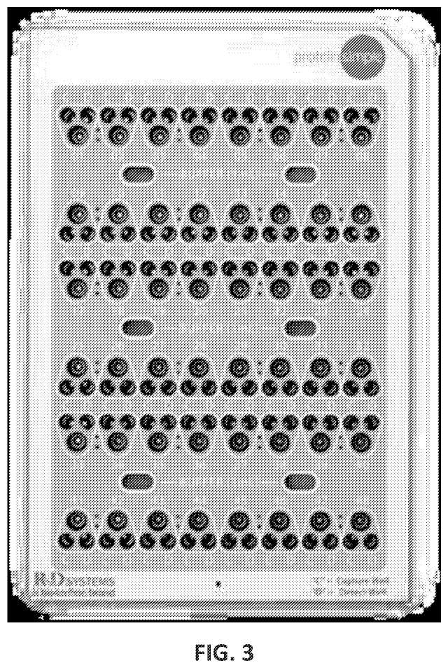

Using Ella Protein Simple's 48-sample “open cartridge” platform (FIG. 3) which allows for the use of any combination of sample, capture and detection antibodies, we optimized our assay to detect SARS-CoV-2 with high sensitivity and precision. We evaluated 40 antibodies in different combinations and concentrations in 45 open cartridges. We evaluated recombinant N, S, M, E proteins as well as wild type virus, heat-inactivated and gamma-irradiated viruses. We also evaluated a number of inactivating agents, assay buffers and collection media. Our assay is now optimized with respect to the antibody pair, antibody concentrations, collection media and inactivating agents.

Using a relative fluorescence intensity (RFU) cutoff of 0.28, our limit of detection (LOD) using recombinant N-protein is 11 μg/mL, and 25,000 RNA copies/mL using gamma-irradiated SARS-CoV-2 from BEI Resources. BEI Resources is the biologic pathogen repository established by the NIH and provides SARS-CoV-2 virus quantified by droplet digital PCR (ddPCR). We have confirmed our LOD using two different lots of gamma-irradiated virus from BEI. Our analytical sensitivity is superior to existing rapid antigen tests by a wide margin, comparable to the best-in-class point-of-care molecular test (Roche Cobas Liat LOD=5,400 RNA copies/mL) and the median laboratory-based RT-PCR platform (LOD=5,400 RNA copies/mL). Our test has several advantages compared with existing POC molecular tests: improved sensitivity, higher throughput, lower cost per test and quantitative results. Similar to the process for other analytes quantified on Ella, standard curves using gamma-irradiated SARS-CoV-2 from BEI Resources will be run in triplicate at the factory. The standard curves are loaded onto a QR code on the cartridge which is scanned prior to starting the test.

Studies of antibodies identified certain combinations of capture and detection antibodies which generated unexpectedly good results (see, e.g., the Tables below). For example:

N5 as a Detection antibody, 40143-R040 (monoclonal rabbit IgG Clone #040) from Sino Biologicals.

N6 as a Capture antibody, MAB10474 (monoclonal Mouse IgG2B Clone #1035111) from R&D.

N7 as a Capture antibody (N7C): R&D Systems Clone 1035145 Catalog #MAB104741

N11 as a Detection antibody (N11D): R&D Systems Clone 1035143 Catalog #MAB104742

From these studies we have discovered that N7C with N11D produce strong signals for positive samples (SP34, SP36, SP37, SP51, SP52), and lowest signals for the negative samples (P129, P155, P170). The negative samples were handpicked for this comparison since they previously showed high background signals. From these studies we also discovered that N7C with N11D produced the strongest signals for gamma-irradiated SARS-CoV-2 virus. From these studies we also discovered that N7C with N11 D produced the lowest background signals for samples without SARS-CoV-2 virus. From these studies we also discovered that N7C (capture) with N11D (detection) is the optimal antibody pair. The N6C (capture) with N11D (detection) antibody pair also works well, although with slightly higher background signals.

The RFU cutoff value for a positive test can be 0.28 RFU. This should give a LOD of around ˜25,000 RNA copies/mL based on cart 44. In such assays it is recommended to try to get the background RFU down as much as possible, at the expense of signal boost for positive samples. Most positive samples will have very high RFUs (median RFU was 35.4 for positive samples), the samples with lower concentrations will be more difficult to diagnose correctly.

We conducted a clinical validation study of our assay among patients at UCLA Ronald Reagan Medical Center, UCLA Santa Monica Medical Center and students at the UCLA Ashe Center for Student Health. We collected over 600 nasal swab samples with 80 SARS-CoV-2 positive samples by RT-PCR. The preliminary analysis on a subset of these samples was run on the Ella ProteinSimple “open cartridge” and showed excellent performance characteristics: Sensitivity 96% (43/45) and specificity 97% (28/29) (Table 3). Median RFU on positive samples (by RT-PCR) was 35.4 with a viral concentration of 5.1 million RNA copies/mL. Negative samples had a median RFU of only 0.1.

These studies validated the surprising sensitivities of the assays disclosed herein.

### REFERENCES

1. Pilarowski G, Lebel P, Sunshine S, Liu J, Crawford E, Marquez C, Rubio L, Chamie G, Martinez J, Peng J, Black D, Wu W, Pak J, Laurie M T, Jones D, Miller S, Jacobo J, Rojas S, Rojas S, Nakamura R, Tulier-Laiwa V, Petersen M, Havlir D V, DeRisi J. Performance characteristics of a rapid SARS-CoV-2 antigen detection assay at a public plaza testing site in San Francisco. medRxiv Prepr Serv Heal Sci 2020; 415-418.doi:10.1101/2020.11.02.20223891.
2. Mühlemann B, Zuchowski M, Karen W, Lei J. Comparison of seven commercial SARS-CoV-2 rapid Point-of-Care Antigen Tests. 2020;
3. Dinnes J, Deeks J J, Adriano A, Berhane S, Davenport C, Dittrich S, Emperador D, Takwoingi Y, Cunningham J, Beese S, Dretzke J, Ferrante di Ruffano L, Harris I M, Price M J, Taylor-Phillips S, Hooft L, Leeflang M M G, Spijker R, Van den Bruel A. Rapid, point-of-care antigen and molecular-based tests for diagnosis of SARS-CoV-2 infection. Cochrane Database Syst Rev 2020; 2020.
4. Drosten C, Corman V. How sensitive are antigen point of care tests towards the end of the first week of symptoms? 2020; at <https://virologie-ccm.charite.de/fileadmin/user_upload/microsites/m_cc05, virologie-ccm/dateien_upload/20201208-AgPOCT_Preprints.pdf>.

All publications mentioned herein (e.g., those disclosed above) are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

TABLE 1
Best Performing Antibodies

| | | | | |
|---|---|---|---|---|
| N6C | R&D | Nucleocapsid Mab (Clone 1035111) | MAB10474 | Capture Ab |
| N7C | R&D | Nucleocapsid Mab (Clone 1035145) | MAB104741 | Capture Ab |
| N5D | Sino | Nucleocapsid Mab (Clone 040) | 40143-R040 | Detection Ab |
| N11D | R&D | Nucleocapsid Mab (Clone 1035143) | MAB104742 | Detection Ab |

TABLE 2
Recombinant N-Protein and Gamma-Irradiated SARS-
CoV-2 Virus Signals for Antibody Combinations

| | | | | | | | |
|---|---|---|---|---|---|---|---|
| | Sample | Capture | Detection | Mean | RFU | | |
| Sample | Description | Ab | Ab | RFU | % CV | avg | S/N |

| | | | | | | |
|---|---|---|---|---|---|---|
| unspiked DA | N6C3.5 | N5D5 | 0.10 | 5.02 | | |
| unspiked DA | N6C3.5 | N5D5 | 0.09 | 0.06 | 0.10 | |
| 10,000G DA | N6C3.5 | N5D5 | 0.12 | 1.83 | | 1.3 |
| 100,000G DA | N6C3.5 | N5D5 | 0.45 | 2.12 | | 4.7 |
| 1,000,000G DA | N6C3.5 | N5D5 | 3.08 | 1.58 | | 32.4 |
| N-prot 32 | N6C3.5 | N5D5 | 0.63 | 1.33 | | 6.6 |
| N-prot 800 | N6C3.5 | N5D5 | 11.95 | 1.86 | | 125.8 |
| Sino N-prot 800 | N6C3.5 | N5D5 | 9.44 | 1.83 | | 99.4 |
| unspiked DA | N6C3.5 | N11D0.4 | 0.12 | 5.05 | | |
| unspiked DA | N6C3.5 | N11D0.4 | 0.14 | 8.49 | 0.13 | |
| 10,000G DA | N6C3.5 | N11D0.4 | 0.14 | 7.07 | | 1.1 |
| 100,000G DA | N6C3.5 | N11D0.4 | 0.48 | 6.05 | | 3.7 |
| 1,000,000G DA | N6C3.5 | N11D0.4 | 3.78 | 1.48 | | 29.1 |
| N-prot 32 | N6C3.5 | N11D0.4 | 0.71 | 2.09 | | 5.5 |
| N-prot 800 | N6C3.5 | N11D0.4 | 17.98 | 2.41 | | 138.3 |
| Sino N-prot 800 | N6C3.5 | N11D0.4 | 0.14 | 2.45 | | 1.1 |
| unspiked DA | N7C3.5 | N11D0.4 | 0.09 | 0.42 | | |
| unspiked DA | N7C3.5 | N11D0.4 | 0.09 | 3.70 | 0.09 | |
| 10,000G DA | N7C3.5 | N11D0.4 | 0.11 | 0.07 | | 1.2 |
| 100,000G DA | N7C3.5 | N11D0.4 | 0.44 | 6.49 | | 4.9 |
| 1,000,000G DA | N7C3.5 | N11D0.4 | 3.97 | 0.87 | | 44.1 |
| N-prot 32 | N7C3.5 | N11D0.4 | 0.80 | 2.68 | | 8.9 |
| N-prot 800 | N7C3.5 | N11D0.4 | 17.82 | 2.78 | | 198.0 |
| Sino N-prot 800 | N7C3.5 | N11D0.4 | 0.13 | 8.05 | | 1.4 |

TABLE 3
Summary of RFU and Viral Concentration
for SARS-CoV-2 Positive Samples

| | | RFU [1] | S/N [2] | Concentration [3] | | |
|---|---|---|---|---|---|---|
| | n | Median | Median | Median | Min | Max |
| Negative pts: | 29 | 0.1 | 1.4 | — | — | — |
| Positive Pts: | 45 | 35.4 | 490.3 | 5,066,041 | 9,013 | 343,280,937 |

Preliminary Performance in this limited panel compared with RT-PCR:
Sensitivity: 43/45 (96%)
Specificity: 28/29 (97%)
[1] Relative fluorescence intensity.
[2] Signal to noise ratio.
[3] RNA Copies/mL.

TABLE 4
Median RFU and Signal to Noise Ratios for
a Subset of Clinical Nasopharyngeal Samples

| | Sample | Capture | Detection | Mean | RFU % | avg |
|---|---|---|---|---|---|---|
| Sample | Description | Ab | Ab | RFU | CV | S/N |
| P129 | negative | N6C3.5 | N5D5 | 0.38 | 8.01 | 4.0 |
| P129 | negative | N6C3.5 | N11D0.4 | 0.39 | 1.92 | 3.0 |
| P129 | negative | N7C3.5 | N11D0.4 | 0.28 | 6.63 | 3.1 |
| SP51 | positive 1 | N6C3.5 | N5D5 | 4.08 | 2.85 | 42.9 |
| SP52 | positive 2 | N6C3.5 | N5D5 | 1.29 | 3.05 | 13.6 |
| SP51 | positive 1 | N6C3.5 | N11D0.4 | 14.29 | 2.83 | 109.9 |
| SP52 | positive 2 | N6C3.5 | N11D0.4 | 3.25 | 5.30 | 25.0 |
| SP51 | positive 1 | N7C3.5 | N11D0.4 | 29.26 | 0.99 | 325.1 |
| SP52 | positive 2 | N7C3.5 | N11D0.4 | 4.17 | 0.49 | 46.3 |

1. A method of detecting SARS-CoV-2 in a fluid sample comprising:
combining the fluid sample with a capture antibody that binds to a SARS-CoV-2 peptide, wherein the capture antibody is coupled to a matrix;
combining the fluid sample with a detection antibody that binds to a SARS-CoV-2 peptide, wherein the detection antibody is coupled to a detectable label; and
observing the presence of SARS-CoV-2 bound by both the capture antibody and the detection antibody such that the presence of SARS-CoV-2 in the fluid sample is detected;
wherein the capture antibody and the detection antibody are selected from the antibodies that bind to epitopes on SARS-CoV-2 bound by N6 antibody, N7 antibody, N11 antibody and/or N5 antibody.

2. The method of claim 1, wherein:
N7 and N11 are the capture and/or the detection antibodies;
N6 and N11 are the capture and/or the detection antibodies; and/or
N6 and N5 are the capture and/or the detection antibodies.

3. The method of claim 1, wherein the method is performed in an assay device comprising microfluidic channels and the capture antibody is coupled to one or more regions within the microfluidic channels.

4. The method of claim 3, wherein:
the fluid sample is directed by pneumatic pistons and valves into a selected region comprising the capture antibody coupled to a matrix within the microfluidic channel and adapted to bind the SARS-CoV-2 peptide;
the detection antibody binds SARS-CoV-2 peptides within the channel that are bound by the capture antibody; and
amounts of detection antibody are quantified by a detectable label comprising at least one of: a fluorochrome, a fluorophore, an enzyme, or a nucleic acid.

5. The method of claim 1, wherein:
amounts of SARS-CoV-2 peptides detected in the fluid sample are quantified using a standard curve with a known concentration of SARS-CoV-2 peptide, wherein the standard curve includes inactivated SARS-CoV-2 and at least one recombinant SARS-CoV-2 protein; and/or
results of the method are characterized quantitatively as RNA copies per milliliter or viral copies per milliliter.

6. The method of claim 1, wherein the method can detect less than 25,000 SARS-CoV-2 RNA copies per milliliter in the fluid sample.

7. The method of claim 1, wherein the detection antibody is present in concentrations between about 0.5-200 μg/mL the capture antibody is present in concentrations of about 0.5-200 μg/mL.

8. The method of claim 1, wherein the fluid sample is combined with inactivating reagents, lysing reagents and/or detergents so as to inactivate SARS-CoV-2 such that peptides are available for binding prior to detecting the presence of SARS-CoV-2 in the fluid sample.

9. A system for detecting the presence of SARS-CoV-2 in a fluid sample, the system comprising:
a detection antibody that binds to a SARS-CoV-2 peptide, wherein the detection antibody is coupled to a detectable label; and
a capture antibody that binds to a SARS-CoV-polypeptide, wherein the capture antibody is coupled to a matrix;
wherein the capture antibody and the detection antibody are selected from the antibodies that bind to epitopes on the SARS-CoV-2 that are bound by N6 antibody, N7 antibody, N11 or N5 antibody.

10. The system of claim 9, wherein:
N7 and N11 are the capture and/or the detection antibodies;
N6 and N11 are the capture and/or the detection antibodies; and/or
N6 and N5 are the capture and/or the detection antibodies.

11. The system of claim 9, wherein the method is performed in an assay device comprising microfluidic channels and the capture and detection antibodies are coupled to one or more regions within the microfluidic channels.

12. The system of claim 11, wherein:
the fluid sample is directed by pneumatic pistons and valves into a selected region where the capture antibody coupled to a matrix within the microfluidic channel captures the SARS-CoV-2 peptide;
the detection antibody binds SARS-CoV-2 peptides within the channel that are bound by the capture antibody; and
amounts of detection antibody are quantified by a detectable label comprising at least one of: a fluorochrome, a fluorophore, an enzyme, or a nucleic acid.

13. The system of claim 9, wherein:
amounts of SARS-CoV-2 peptides detected in the fluid sample are quantified using a standard curve with a known concentration of SARS-CoV-2 peptide, wherein the standard curve includes inactivated SARS-CoV-2 and at least one recombinant SARS-CoV-2 protein; and/or
results are characterized quantitatively as RNA copies per milliliter or viral copies per milliliter.

14. The system of claim 9, wherein the detection antibody is present in concentrations between about 0.5-200 μg/mL and the capture antibody is present in concentrations of about 0.5-200 μg/mL

15. The system of claim 9, the detection antibody is present in concentrations between about 1-10 μg/mL and the capture antibody is present in concentrations of about 1-10 μg/mL.

16. The system of claim 9, wherein the fluid sample is combined with inactivating reagents, lysing reagents and/or detergents so as to inactivate SARS-CoV-2 such that SARS-CoV-2 peptide epitopes are available for binding prior to detecting the presence of SARS-CoV-2 in the fluid sample.

17. The system of claim 9, wherein the system can detect less than 25,000 SARS-CoV-2 RNA copies per milliliter in the fluid sample.

18. The system of claim 17, wherein the system can detect SARS-CoV-2 present in the sample at concentrations of less than 10,000 SARS-CoV-2 viral particles (or RNA copy numbers) per milliliter of fluid tested.

19. A kit comprising at least two of: N6 antibody, N7 antibody, N11 antibody and N5 antibody.

20. The kit of claim 19, further comprising at least one of inactivating reagents, lysing reagents and detergents.
